(19) United States
(12) Patent Application Publication
(10) Pub. No.: US 2022/0387660 A1
(43) Pub. Date: Dec. 8, 2022

(54) OXYGEN-GENERATING WOUNDING HEALING DRESSING

(71) Applicant: University of Florida Research Foundation, Inc., Gainesville, FL (US)

(72) Inventors: Nicholas J. Abuid, Gainesville, FL (US); Jiapu Liang, Gainesville, FL (US); Cherie Stabler, Gainesville, FL (US)

(21) Appl. No.: 17/772,709

(22) PCT Filed: Nov. 6, 2020
(86) PCT No.: PCT/US2020/059390

§ 371 Date: Apr. 28, 2022

Related U.S. Application Data
(60) Provisional application No. 63/088,015, filed on Oct. 6, 2020.
(60) Provisional application No. 62/942,422, filed on Dec. 2, 2019.

(51) Int. Cl. A61L 15/44

(52) U.S. Cl. CPC A61L 15/44; A61L 2300/102; A61L 2300/41; A61L 2300/412; A61L 2300/11

(57) ABSTRACT

The present disclosure provides for oxygen generating and free radical scavenging biomaterials, an article including the oxygen generating and free radical scavenging biomaterial, wound healing dressings or structures (e.g., bandage) that have the characteristic of generating oxygen and having redox modulating capabilities. In particular, the wound healing dressing can have the characteristics of being anti-inflammatory, antioxidant, and pro-healing for extended periods of time (e.g., at least 10 days, at least 30 days).

### CROSS-REFERENCE TO RELATED APPLICATIONS

This application claims the benefit of and priority to U.S. Provisional Application Ser. No. 62/942,422, having the title “OXYGEN GENERATING AND FREE RADICAL SCAVENGING BIOMATERIAL” filed on Dec. 2, 2019, the disclosure of which is incorporated herein in by reference in its entirety.

In addition, this application claims the benefit of and priority to U.S. Provisional Application Ser. No. 63/088,015, having the title “OXYGEN GENERATING AND FREE RADICAL SCAVENGING BIOMATERIAL” filed on Oct. 6, 2020, the disclosure of which is incorporated herein in by reference in its entirety.

### STATEMENT REGARDING FEDERALLY SPONSORED RESEARCH OR DEVELOPMENT

This invention was made with government support under 5R01DK100654-05 awarded by the National Institutes of Health. The government has certain rights in the invention.

### NON-FEDERAL FUNDING SUPPORT

This invention was made in part or in whole with support under Grant Nos. 3-SRA-2015-38-Q-R and 3-SRA-2018-683-S-B, awarded by the Juvenile Diabetes Research Foundation.

### BACKGROUND

The number of diabetic patients have quadrupled in the last thirty years, and 1 out of 11 people in the world have diabetes.[1] About 15% of diabetic patients will develop diabetic foot ulcers (DFUs), indicating an increasing need for a product that can help with the wound healing.[2]

### SUMMARY

Embodiments of the present disclosure provide for oxygen generating and free radical scavenging biomaterials, an article including the oxygen generating and free radical scavenging biomaterial, wound healing dressings or structures (e.g., bandage) that have the characteristic of generating oxygen and having redox modulating capabilities. The oxygen generating and free radical scavenging biomaterial can be used in an article such as a wound healing dressing, bandage, or the like. The oxygen generating and free radical scavenging biomaterial can be used by itself as a paste, fluid, or otherwise and directly applied as needed. The In an aspect, the present disclosure provides for a wound healing dressing, comprising a substrate having a first side and a second side on the side opposite the first side, wherein the second side is adjacent the wound, wherein at least a first medicant layer is disposed on the second side of the substrate, wherein the first medicant layer has a first side adjacent the second side of the substrate and the first medicant layer has a second side opposite the first side of the first layer, the first side on the side opposite the wound, wherein the first medicant layer comprises one or more of the following: oxygen-generating composite material and a cerium oxide material.

In an aspect, the present disclosure provides for a oxygen generating and free radical scavenging biomaterials (e.g., wound healing dressing), comprising a substrate having a first side and a second side on the side opposite the first side, wherein the second side is adjacent the wound, wherein at least a first medicant layer is disposed on the second side of the substrate, wherein the first medicant layer has a first side adjacent the second side of the substrate and the first medicant layer has a second side opposite the first side of the first layer, the second side adjacent the wound, wherein the first medicant layer comprises one or more of the following: oxygen-generating composite material and a cerium oxide material, wherein the oxygen-generating composite material is a flexible sheet that has a thickness of about 200 to 1000 μm, wherein the flexible sheet has a length of about 1 centimeter to 10 centimeters and a width of about 1 centimeter to 10 centimeters, and wherein the flexible sheet generates greater than about 0.32 mM/day for at least 5 days.

In an aspect, the present disclosure provides for a oxygen generating and free radical scavenging biomaterials (e.g., wound healing dressing), comprising a substrate having a first side and a second side on the side opposite the first side, wherein the second side is adjacent the wound, wherein a first medicant layer is disposed on the second side of the substrate, wherein the first medicant layer has a first side adjacent the second side of the substrate and the first medicant layer has a second side opposite the first side of the first layer, wherein the substrate includes a second medicant layer, wherein the second medicant layer has a first side and a second side opposite the first side, wherein the first side of the second medicant layer is adjacent the second side of the first medicant layer, wherein the first medicant layer comprises one or more of the following: an oxygen-generating composite material and a cerium oxide material, and wherein the second medicant layer comprises one or more of the following: the oxygen-generating composite material and the cerium oxide material.

### BRIEF DESCRIPTION OF THE DRAWINGS

Further aspects of the present disclosure will be more readily appreciated upon review of the detailed description of its various embodiments, described below, when taken in conjunction with the accompanying drawings.

FIG. 1 illustrates schematics of oxygen-generating and free radical scavenging biomaterial wound healing dressing designs. FIG. 1A illustrates CONPs mixed with alginate to form a free radical scavenging layer, which will directly contact the wound and scavenge free radicals generated from both wound area and the oxygen-generating composite (O₂GC) layer, which releases oxygen. FIG. 1B illustrates CONPs mixed within the oxygen-generating composite (O₂GC) layer to scavenge free radicals, while this layer releases oxygen simultaneously. FIG. 1C illustrates CONPs mixed with alginate and oxygen-generating composite (O₂GC) microbeads to form a layer, where CONPS scavenging surrounding free radicals and oxygen-generating composite (O₂GC) microbeads release oxygen.

FIG. 2 illustrates the fabrication of the oxygen-generating composite layer using a silicon mold

FIG. 3 illustrates the oxygen release profiles from oxygen-generating composite layer with different thickness

FIG. 4 illustrates glass (top) and alginate (bottom) beads coated with alternating layers of CONP and fluorescently labeled alginate (Scale=200 μm).

FIG. 5 illustrates bright field microscopy of coated alginate beads (FIG. 5A) show different coating morphology depending on the formulation used. Parameters such as molecular weight of alginate and pH of CONP during layering were manipulated for controlled thickness (FIGS. 5B and 5C) and uniformity (FIGS. 5D and 5E). (*: p=0.05, **: p=0.01, ***: p=0.001, ****: p=0.0001; γ: significance difference from CONP₄ analog; Δ: significant difference from six-layer group).

FIG. 6 illustrates cerium content on coated microbeads was quantified via ICP-MS.

FIG. 7 illustrates that as the conditions of the coatings are changed, the multi-enzymatic activity is also altered. A thorough investigation in which a combination of parameters such as alginate MW and CONP pH were interchanged to achieve a repertoire of coating formulations with varying catalase-to-SOD activity ratio. (*: p<0.05, **: p<0.01, ***: p<0.001, ****: p<0.0001)

FIG. 8 illustrates that as the catalase-mimetic activity of the coatings was assessed by challenging coated alginate beads to concentrations of H₂O₂ characteristic of an inflammatory microenvironment. (Statistical analysis (*: p<0.05, **: p<0.01, ***: p<0.001, ****: p<0.0001; γ: significance difference from CONP₄ analog; A: significant difference from six-layer group)

FIG. 9 illustrates β-cells encapsulated in alginate beads were exposed to H₂O₂ or superoxide (Xa/XO). When compared to non-coated beads (ROS damage represented as a solid black line), twelve-layer coatings were able to protect (1A) more efficiently than six-layer coatings (1B). β-cell protection from H₂O₂ or superoxide (Xa/XO) is dependent on the catalase/SOD ratio of each formulation. (*: p<0.05, **: p<0.01, ***: p<0.001, ****: p<0.0001; γ: significance difference from No ROS control)

FIG. 10 illustrates CONP₄/MVG coatings on PDMS disks can decrease the inflammatory response of on LPS-activated macrophages.

### DETAILED DESCRIPTION

This disclosure is not limited to particular embodiments described, and as such may, of course, vary. The terminology used herein serves the purpose of describing particular embodiments only, and is not intended to be limiting, since the scope of the present disclosure will be limited only by the appended claims.

Where a range of values is provided, each intervening value, to the tenth of the unit of the lower limit unless the context clearly dictates otherwise, between the upper and lower limit of that range and any other stated or intervening value in that stated range, is encompassed within the disclosure. The upper and lower limits of these smaller ranges may independently be included in the smaller ranges and are also encompassed within the disclosure, subject to any specifically excluded limit in the stated range. Where the stated range includes one or both of the limits, ranges excluding either or both of those included limits are also included in the disclosure.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure. Any recited method may be carried out in the order of events recited or in any other order that is logically possible.

Embodiments of the present disclosure will employ, unless otherwise indicated, techniques of, chemistry, biology, and the like, which are within the skill of the art. Such techniques are explained fully in the literature.

Unless otherwise defined, all technical and scientific terms used herein have the same meaning as commonly understood by one of ordinary skill in the art of material science, chemistry, textiles, polymer chemistry, and the like. Although methods and materials similar or equivalent to those described herein can be used in the practice or testing of the present disclosure, suitable methods and materials are described herein.

As used in the specification and the appended claims, the singular forms “a,” “an,” and “the” may include plural referents unless the context clearly dictates otherwise. Thus, for example, reference to “a support” includes a plurality of supports. In this specification and in the claims that follow, reference will be made to a number of terms that shall be defined to have the following meanings unless a contrary intention is apparent.

### DISCUSSION

The present disclosure provides for an oxygen generating and free radical scavenging biomaterials, an article including the oxygen generating and free radical scavenging biomaterial, wound healing dressings or structures (e.g., bandage) that have the characteristic of generating oxygen and having redox modulating capabilities. In particular, the oxygen generating and free radical scavenging biomaterials and the wound healing dressing can have the characteristics of being anti-inflammatory, antioxidant, and pro-healing (e.g., making or becoming healthy again from a diseased or damaged state) for extended periods of time (e.g., at least 10 days, or at least 30 days). The structures can be applied to wounds such as diabetic foot ulcers (DFUs) to provide enough oxygen for tissue regeneration and redox modulation to combat tissue damage caused by chronic inflammation. The oxygen generating and free radical scavenging biomaterials or wound healing dressing can provide a steady release of oxygen during this time to promote cell-mediated remodeling of skin. Additionally, as hard-to-treat wounds commonly exhibit oxidative, noxious environments that are unsupportive of angiogenic and pro-healing factors, the patch provides antioxidant support to combat oxidative damage in the wound site and modulate the immune cell phenotype from a pro-inflammatory to an anti-inflammatory state. In other words, the present disclosure provides for oxygen generating and free radical scavenging biomaterials or wound healing dressing that provide the dual benefits of localized oxygen delivery and scavenging of ROS (a significant detrimental by-product in wound healing and lots of inflammatory conditions). Further, this embodiment may enhance other therapeutic interventions (e.g. exogenous growth factors) by mitigating oxidant-mediated degradation and support host cell survival.

Other products do not provide these characteristics for extended periods of time. In particular, other products may release too much oxygen too quickly in a manner that harms the tissue and exacerbates oxidative stress, in a way that results in cell death. In contrast, the present disclosure can tailor the release of oxygen and can modulate redox reactions over an extended period of time so that the tissue can be healed.

In an aspect, the oxygen generating and free radical scavenging biomaterials or the wound healing dressing includes an oxygen-generating composite material and a cerium oxide material. Each of these are provided in effective amounts to accomplish the desired result, where the effective amount can be modulated for different types of wounds, different people, the desired outcome, and the like. As used herein, the term “effective amount” refers to the amount of each needed that is sufficient to effect beneficial or desired results, including clinical results. When used with reference to these components, “effective amount” refers to the amount necessary to permit cause the wound to heal as desired.

In an aspect, the oxygen generating and free radical scavenging biomaterials or the wound healing dressing can have the characteristics of being anti-inflammatory, an antioxidant, and pro-healing, where the wound healing dressing can have these characteristics for extended periods of time, for example at least 5 days, at least 10 days, at least 20 days or at least 30 days or more. The oxygen generating and free radical scavenging biomaterials or the wound healing dressing has the characteristics of free radical scavenging and oxygen generation.

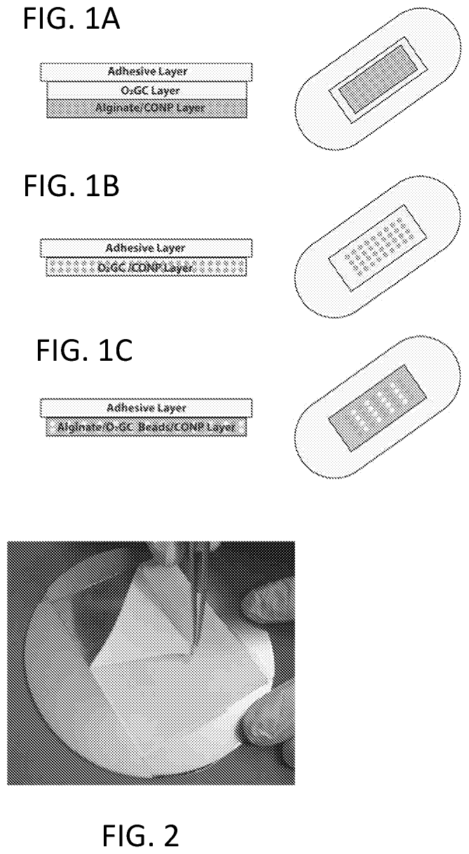

In an aspect, the oxygen generating and free radical scavenging biomaterials or the wound healing dressing includes a substrate having a first side and a second side on the side opposite the first side. The first side is on the side opposite the wound and the second side may be immediately adjacent the wound or adjacent (e.g., a layer may be therebetween) the wound. At least a first medicant layer is disposed on the second side of the substrate and is adjacent the wound. The first medicant layer has a first side adjacent the second side of the substrate and the first medicant layer has a second side opposite the first side of the first layer. The first medicant layer can include one or more of the following: oxygen-generating composite material and a cerium oxide material. In addition, the oxygen generating and free radical scavenging biomaterials or the wound healing dressing can include one or more additional medicant layers adjacent the first and/or second side of the first medicant layer. The first medicant layer, the second medicant layer, or a combination thereof can have the characteristic of free radical scavenging, oxygen generation, or a combination thereof. Additional details are provided herein and in the Example, where FIGS. 1A-1C provide illustrative configurations of the wound healing dressing.

The cerium oxide material can include a plurality of cerium oxide nanoparticles (e.g., 10s of thousands, to millions and more depending upon the surface area (in an effective amount to achieve the desired goal)). Cerium oxide nanoparticles have oxidant scavenging capabilities useful in healing dressings. Cerium oxide nanoparticles (CONPs) are nanometer-sized crystals of cerium oxide, typically having the longest dimension of about 1 to 20 nanometers, about 3 to 15 nanometers, about 3 to 10 nanometers, or about 3 to 5 nanometers. Cerium oxide crystals have a fluorite-type crystal lattice and the cerium atoms are present in +3 or +4 valence states, where the relative amount of each depends on factors include how it is made and the like.

The cerium oxide material also includes at least one biopolymer that can be mixed with the nanoparticles so that the nanoparticles are dispersed within the biopolymer. The biopolymer can be polymers such as alginate, hyaluronic acid (HA), chitosan, agarose, collagen, fibrin, gelatin, dextran, and any combination thereof, as well as derivatives of each of these and combinations thereof. In an aspect, the biopolymer is an alginate or derivatives thereof, where the alginate can have a molecular weight of about 10kDa to 500kDa. The weight ratio of the cerium oxide nanoparticles to the biopolymer can be about 1:99 and 99:1 or about 1:1 to 10:1 or about 80:20 to 20:80 or about 70:30 to 30:70.

The oxygen-generating composite material can include a peroxide material and a support material. The peroxide material can include one or more of the following peroxide materials: calcium peroxide, sodium peroxide, magnesium peroxide, lithium peroxide, potassium peroxide, and a combination thereof. The peroxide materials can be particles such as nanoparticles (e.g., 10 to 900 nm in the longest dimension (e.g., diameter)) or microparticles (e.g., 1 to 100 μm in the longest dimension (e.g., diameter)). The peroxide material can produce oxygen by the reaction of peroxide with water, which produces a hydroxide and hydrogen peroxide. The hydrogen peroxide spontaneously decomposes into water and oxygen. It should be stated that the intermediate hydrogen peroxide product can harm biological material. However, the use of the support encapsulating material can reduce or prevent the hydrogen peroxide intermediate from contacting viable cells (e.g., skin or the like) through both modulating the kinetics of the reaction or delaying or preventing their release from the biomaterial.

The oxygen-generating composite material can include one or more of the following support materials: organosilicones, poly(ethersulfone), poly(ethylene oxide terephthalate) block copolymers, polysulfone, and combinations thereof. The weight ratio of the support material to the peroxide material can range from 1:1 to 99:1 or about 1:1 to 10:1 or about 80:20 to 20:80 or about 70:30 to 30:70, depending on the intended use.

Now having described aspects of the wound healing dressing and components thereof in general, additional aspects are now described. The oxygen-generating composite material, the first medicament layer, and/or the second medicant layer can be a flexible sheet that has a thickness of about 200 to 1000 μm, about 250 to 900 μm, or about 250 to 500 μm. The other dimension such as length and width or diameter can vary depending upon the specific application and are typically in the range of 1 centimeter to 10 centimeters or 100 centimeters or more. When the sheet has a thickness of greater than 1000 μm, the sheet is not flexible and is difficult for application in a wound healing dressing, as flexible materials are more desirable. The flexible sheet including the oxygen-generating composite material can generate about 0.05 to about 1.68 mM/day, about 0.18 to about 2.52 mM/day, or about 0.25 to about 2.65 mM/day for at least 30 days.

As stated above, the wound healing dressing can include one or more medicant layers (e.g., first medicant layer, second medicant layer, and so on). In an example, the first medicant layer can include a plurality of cerium oxide material particles dispersed in a layer of oxygen-generating composite material. Also, the oxygen-generating composite material can include one or more of cerium oxide nanoparticles (e.g., the cerium oxide nanoparticles are supported in the oxygen-generating composite material (e.g., the biopolymer)) or the cerium oxide nanoparticles and the biopolymer.

In another example, the wound healing dressing can include a second medicant layer. The second medicant layer can have a first side and a second side opposite the first side, where the first side of the second medicant layer is adjacent the second side of the first medicant layer. The second medicant layer can include one or more of the following: oxygen-generating composite material and a cerium oxide material. In an aspect, the first medicant layer includes the oxygen-generating composite material, and the second medicant layer includes the cerium oxide nanoparticles dispersed in the biopolymer.

The present disclosure also includes the following features.

Feature 1. An oxygen generating and free radical scavenging biomaterial (e.g., wound healing dressing or other article), comprising a substrate having a first side and a second side on the side opposite the first side, wherein the second side is adjacent the wound, wherein at least a first medicant layer is disposed on the second side of the substrate, wherein the first medicant layer has a first side adjacent the second side of the substrate and the first medicant layer has a second side opposite the first side of the first layer, the second side adjacent the wound, wherein the first medicant layer comprises one or more of the following: oxygen-generating composite material and a cerium oxide material.

Feature 2. The oxygen generating and free radical scavenging biomaterial (e.g., wound healing dressing) of feature 1, wherein the oxygen generating and free radical scavenging biomaterial or the wound healing dressing has the characteristics of being anti-inflammatory, an antioxidant, and pro-healing for at least 5 days, at least 10 days, at least 20 day or at least 30 days.

Feature 3. The oxygen generating and free radical scavenging biomaterial (e.g., wound healing dressing) of any one of features 1 to 2, wherein the oxygen generating and free radical scavenging biomaterial or the wound healing dressing has the characteristics of free radical scavenging and oxygen generation, optionally wherein the first medicant layer, the second medicant layer, or a combination thereof has the characteristic of free radical scavenging, oxygen generation, or a combination thereof.

Feature 4. The oxygen generating and free radical scavenging biomaterial (e.g., wound healing dressing) of any one of features 1 to 3, wherein cerium oxide material comprises a plurality of cerium oxide nanoparticles, optionally wherein the cerium oxide nanoparticle has a longest dimension, such as diameter, of about 1 to 20 nanometers, about 3 to 15 nanometers, about 3 to 10 nanometers, or about 3 to 5 nanometers.

Feature 5. The oxygen generating and free radical scavenging biomaterial (e.g., wound healing dressing) of any one of features 1 to 4, wherein the cerium oxide material further comprises at least one biopolymer selected from alginate, hyaluronic acid (HA), chitosan, agarose, collagen, fibrin, gelatin, dextran, and any combination thereof, as well as derivatives of each of these; optionally wherein the weight ratio of the cerium oxide nanoparticles to the biopolymer is about 1:99 and 99:1 or about 1:1 to 10:1.

Feature 6. The oxygen generating and free radical scavenging biomaterial (e.g., wound healing dressing) of any one of features 1 to 5, wherein the biopolymer is a hydrogel or derivatives thereof, optionally wherein the alginate has a molecular weight of about 10 kDa to 500 kDa.

Feature 7. The oxygen generating and free radical scavenging biomaterial (e.g., wound healing dressing) of any one of features 1 to 6, wherein the oxygen-generating composite material comprises one or more of the following peroxide materials: calcium peroxide, sodium peroxide, magnesium peroxide, lithium peroxide, potassium peroxide, and a combination thereof; and optionally wherein the oxygen-generating composite material further comprises a support material, wherein the support materials comprises: organosilicones, poly(ethersulfone), poly(ethylene oxide terephthalate) block copolymers, polysulfone, and combinations thereof; optionally wherein the weight ratio of the support material to the peroxide material is about 1:1 to 99:1 or about 1:1 to 10:1.

Feature 8. The oxygen generating and free radical scavenging biomaterial (e.g., wound healing dressing) of any one of features 1 to 7, wherein the oxygen-generating composite material is a flexible sheet that has a thickness of about 200 to 1000 μm, optionally wherein the thickness is about 250 to 900 μm or optionally wherein the thickness is about 250 to 500 μm, optionally the flexible sheet has a length of about 1 centimeter to 10 centimeters and a width of about 1 centimeter to 10 centimeters, optionally wherein the flexible sheet generates about greater than about 0.05 mM/day, greater than about 0.18 mM/day, or greater than about 0.25 mM/day for at least 30 days.

Feature 9. The oxygen generating and free radical scavenging biomaterial (e.g., wound healing dressing) of any one of features 1 to 8, wherein the first medicant layer comprises a plurality of cerium oxide material particles dispersed in a layer of oxygen-generating composite material, optionally wherein the oxygen-generating composite material comprises one or more of cerium oxide nanoparticles and the biopolymer, optionally wherein the oxygen-generating composite material comprises both cerium oxide nanoparticles and the biopolymer, optionally wherein the biopolymer is alginate.

Feature 10. The oxygen generating and free radical scavenging biomaterial (e.g., wound healing dressing) of any one of features 1 to 9, further comprising a second medicant layer, wherein the second medicant layer as a first side and a second side opposite the first side, wherein the first side of the second medicant layer is adjacent the second side of the first medicant layer, wherein the second medicant layer comprises one or more of the following: oxygen-generating composite material and a cerium oxide material.

Feature 11. The oxygen generating and free radical scavenging biomaterial (e.g., wound healing dressing) of any one of features 1 to 10, wherein the first medicant layer includes the oxygen-generating composite material, optionally wherein the oxygen-generating composite material includes a peroxide material, a support material or both, optionally wherein the weight ratio of the support material to the oxygen-generating composite material is about 1:1 to 99:1 or about 1:1 to 10:1; and the second medicant layer includes the cerium oxide material and optionally a biopolymer, optionally wherein the weight ratio of the cerium oxide material to the biopolymer is about 1:1 to 99:1 or about 1:1 to 10:1.

Feature 12. The oxygen generating and free radical scavenging biomaterial (e.g., wound healing dressing) of any one of features 1 to 11, wherein the second medicant layer comprises the cerium oxide nanoparticles dispersed in the biopolymer.

Feature 13. The oxygen generating and free radical scavenging biomaterial (e.g., wound healing dressing) of any one of features 1 to 12, wherein the first medicant layer is a flexible sheet that has a thickness of about 200 to 1000 μm, wherein the flexible sheet has a length of about 1 centimeter to 10 centimeters and a width of about 1 centimeter to 10 centimeters.

Feature 23. The oxygen generating and free radical scavenging biomaterial (e.g., wound healing dressing) of any one of features 1 to 12, wherein the first medicant layer and the second medicant layer form a flexible sheet that has a thickness of about 200 to 1000 μm, wherein the flexible sheet has a length of about 1 centimeter to 10 centimeters and a width of about 1 centimeter to 10 centimeters.

Additional features and descriptions are provided in the Examples.

### EXAMPLE

This disclosure describes a wound healing dressing using oxygen-generating biomaterial with free radical scavenging nanoparticles. This dressing will be manufactured into a small patch size that can be applied to the patient's wound and replaced every 5-15 days. This patch will release sufficient oxygen to support cellular remodeling of skin; it can also provide antioxidant support to combat oxidative damage in the wound site and modulate the immune cell phenotype from a pro-inflammatory to an anti-inflammatory state.

The example provides for an oxygen-generating wound dressing with the ability of free radical scavenging. This can be achieved by using the combination of oxygen-generating composite and cerium oxide nanoparticles (CONP); a self-renewable, ubiquitous, free radical scavenger). Several application prototypes designs, but not limited to, are shown in FIG. 1. The shape of this combination product can be easily optimized for different wounds besides DFUs. The biomaterial itself can also be utilized for a broad range of applications other than wound healing, such as supporting cellular transplantation.

Calcium peroxide was loaded within PDMS to make oxygen-generating composite, as described previously.[3] A silicon mold with different depth (250, 500, and 1000 μm) was used to fabricate the oxygen-generating composite layers (FIG. 2). To characterize release, 10 mm (in diameter) oxygen-generating composite disks of different thickness were immersed in 1 mL PBS solution at 37 ° C. for a 30-day in vitro release study. The kinetic release of oxygen was recorded by an optical sensor in a sealed chamber. Coatings of CONP and alginate were done by first submerging biomaterials such as alginate microbeads, PDMS disks, or stainless-steel wires in 3 mg/mL dispersion of CONP or solution of alginate for thirty seconds followed by three washes with MOPS buffer in between layers.

The cerium concentration on microbeads was analyzed via Induced Coupled Plasma—Mass Spectrometry (ICP-MS) and the catalase/SOD-mimetic activity ratio was acquired by coating stainless-steel electrodes and acquiring the reduction and oxidation potentials via cyclic voltammetry.

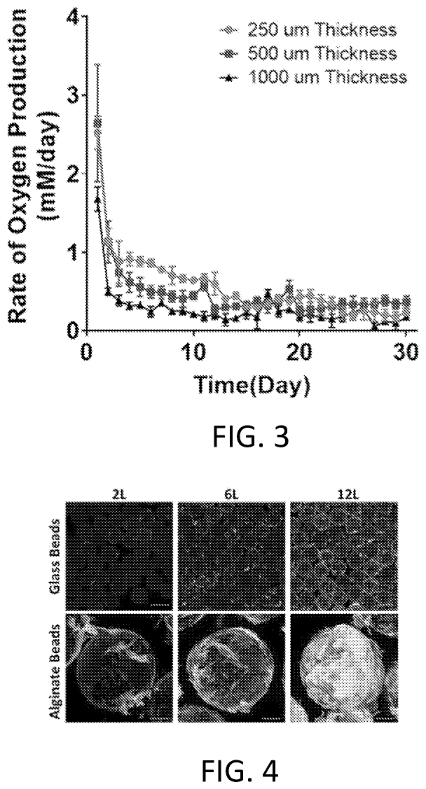

The oxygen-generating composite layer can be fabricated with the thickness from 250 μm to 1000 μm. The measured in vitro release profile indicates that oxygen-generating composite layer of 250, 500, and 1000 μm thickness could generate oxygen >0.18, 0.25, and 0.05 mM per day for over 30 days, respectively (FIG. 3). These findings were unexpected, as drug delivery platforms typically alter release profiles when the geometric scales are modified. With this flexibility, we were unexpectedly able to transition from a thin, non-flexible disk (1000 μm thickness) to a thin (250 um) and highly flexible sheet that is easily adaptable for use as a bandage. These thin, flexible sheets retained their durable and controlled oxygen release, with durable local oxygen generation for over 1 month.

In parallel to the development of an oxygen-generating material, antioxidant coatings consisting of cerium oxide nanoparticles (CONP) were developed. CONP is a highly unique antioxidant and nanoparticle, as it has broad oxidant scavenging capabilities, tunability in reactivity, self-renewing properties (so its activity does not run out), and high activity per unit volume. While others are exploring the use of CONP for medical applications, most seek to inject or locally deliver free nanoparticles. This approach, while potential beneficial, has the disadvantage of phagocytosis by the cells and potential cytotoxicity (as the cells eat the particles). It is also difficult to control delivery and migration of these particles due to their size. The delivery of nanoparticles has been broadly challenging from a regulatory aspect as well. To avoid these challenges, CONP was integrated into a coating, which can be applied to implants or devices to minimize standard inflammatory responses to these materials. In this manner, the CONP is localized.

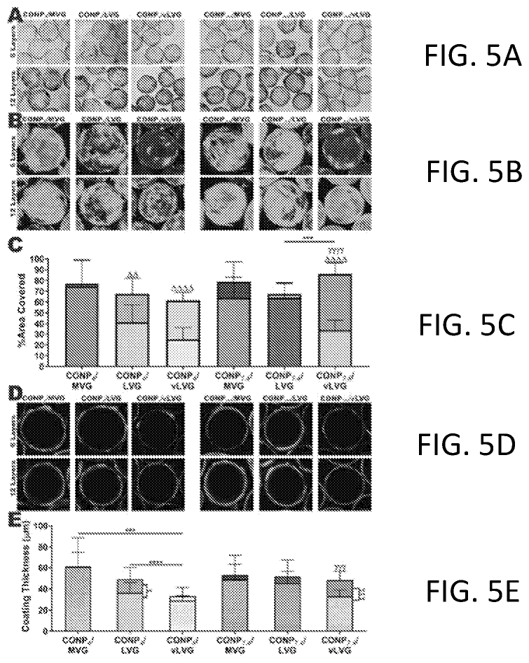

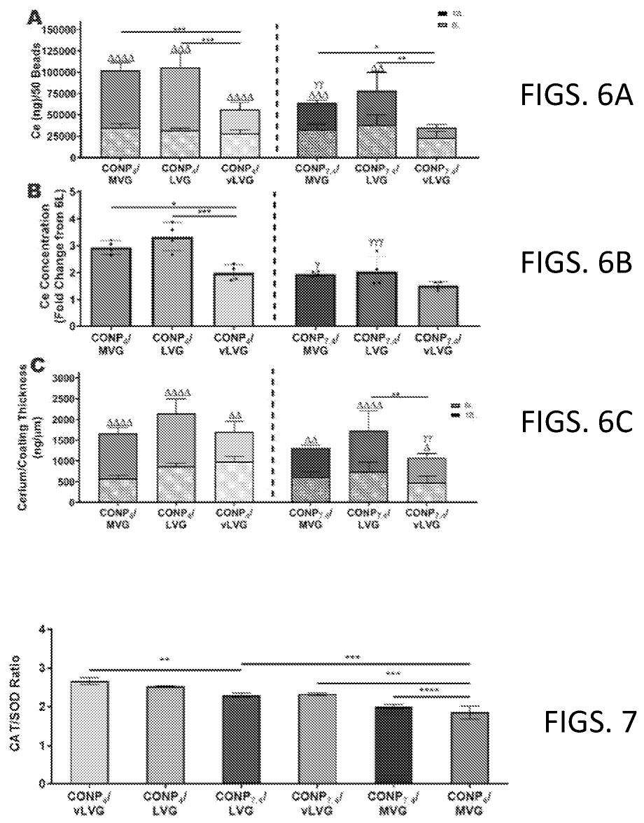

We have demonstrated the ability to create CONP coatings on a wide range of polymeric, metallic, and ceramic biomaterials (FIG. 4). Alginate was used as the layering material to achieve controllable coating uniformity and thickness (FIG. 4). In addition to controlled number of layers, the effect of MW of alginate and the pH of CONP during layering on coating thickness and uniformity was investigated. Tweaking these parameters also allows for controlled thickness and uniformity of surface coating (FIG. 5), as well as the total amount of cerium incorporated within the coatings (FIG. 6).

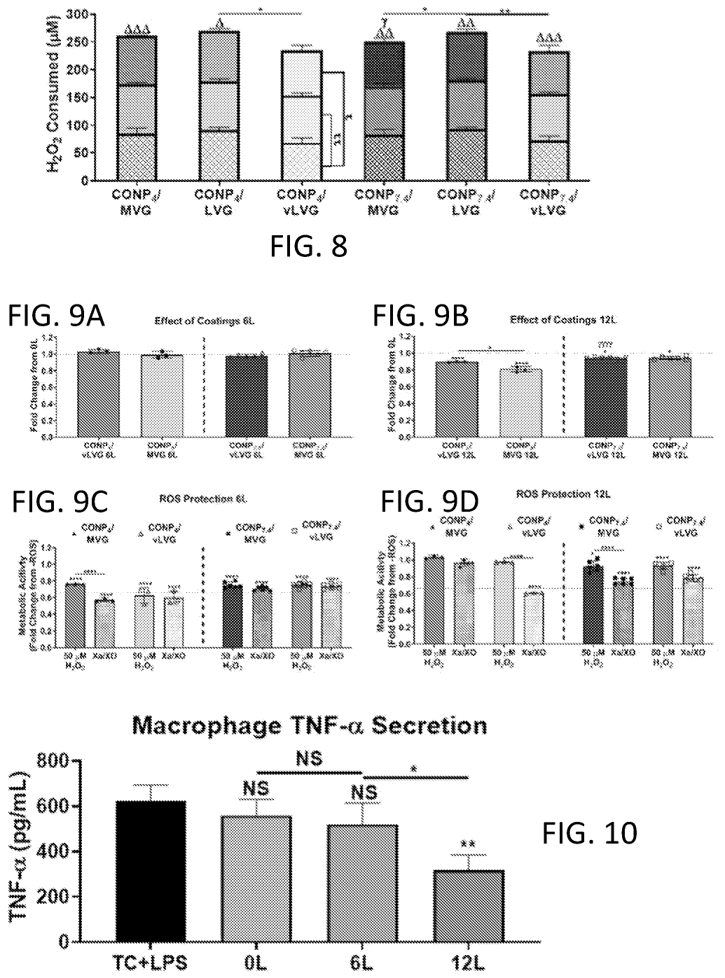

As shown in FIG. 7, different formulations of CONP/alginate coatings can also dictate the multi-enzymatic activity ratio of these coatings. Since, CONP can act as both catalase and SOD, the effect of MW and CONP pH was assessed to understand how different combinations of these two parameters will lead to changes in the ratio of catalase-/SOD-mimetic activity. Understanding the role of these parameters allowed for freedom in choosing the most appropriate formulation for applications in which either H₂O₂ or SO has higher prevalence. Therefore, coated microbeads were exposed to three sequential challenges of 100 μM of H₂O₂, which are concentrations characteristic of inflammatory conditions (FIG. 8).

Interestingly, coatings using alginate vLVG capture lower cerium concentrations than with alginate MVG. However, the amount of H₂O₂ scavenged is comparable due to the increased catalase/SOD activity ratio of vLVG over MVG coatings. The ROS consumption rate and specificity translates to β-cell protection. The ability of this material to protect cells from H₂O₂ as well as SO is dependent on the amount of cerium and the ratio of catalase/SOD activity of each formulation (FIG. 9). This property of the coatings is beneficial in conditions in which inflammation becomes chronic and continuous ROS production leads to cell death and tissue damage. These results also indicate that the formulation can be manipulated when dealing with different types of ROS. This property of the coatings is beneficial when treating inflammatory conditions in which either H₂O₂ or SO is predominant.

Finally, a pilot study was performed in which PDMS disks were coated with CONP and alginate and seeded with activated macrophages which have a pro-inflammatory phenotype. Disks coated with twelve layers of CONP/Alginate were capable of decreasing secretion of pro-inflammatory cytokines by activated macrophages (FIG. 10). The full potential of these coatings to mitigate inflammatory response to biomaterials will be further assessed by in vitro and in vivo implantation.

The oxygen-generating composite layer can be fabricated into different thickness and generate oxygen for over 30 days. Antioxidant coatings including CONPs can scavenge ROS and potentially mitigate inflammatory responses.

### REFERENCES

[1] Y. Zheng, S.H. Ley, F.B. Hu, Global aetiology and epidemiology of type 2 diabetes mellitus and its complications, Nat Rev Endocrinol 14(2) (2018) 88-98.
[2] L. Yazdanpanah, M. Nasiri, S. Adarvishi, Literature review on the management of diabetic foot ulcer, World J Diabetes 6(1) (2015) 37-53.
[3] E. Pedraza, M.M. Coronel, C.A. Fraker, C. Ricordi, C.L. Stabler, Preventing hypoxia-induced cell death in beta cells and islets via hydrolytically activated, oxygen-generating biomaterials, Proc Natl Acad Sci USA 109(11) (2012) 4245-50.

It should be noted that ratios, concentrations, amounts, and other numerical data may be expressed herein in a range format. It is to be understood that such a range format is used for convenience and brevity, and thus, should be interpreted in a flexible manner to include not only the numerical values explicitly recited as the limits of the range, but also to include all the individual numerical values or sub-ranges encompassed within that range as if each numerical value and sub-range is explicitly recited. To illustrate, a concentration range of “about 0.1 percent to about 5 percent” should be interpreted to include not only the explicitly recited concentration of about 0.1 weight percent to about 5 weight percent, but also include individual concentrations (e.g., 1 percent, 2 percent, 3 percent, and 4 percent) and the sub-ranges (e.g., 0.5 percent, 1.1 percent, 2.2 percent, 3.3 percent, and 4.4 percent) within the indicated range. In an aspect, the term “about” can include traditional rounding according to significant figures of the numerical value. In addition, the phrase “about ‘x’ to ‘y’” includes “about ‘x’ to about ‘y’”.

Many variations and modifications may be made to the above-described aspects. All such modifications and variations are intended to be included herein within the scope of this disclosure and protected by the following claims.

1. An wound healing dressing, comprising a substrate having a first side and a second side on the side opposite the first side, wherein the second side is adjacent the wound, wherein at least a first medicant layer is disposed on the second side of the substrate, wherein the first medicant layer has a first side adjacent the second side of the substrate and the first medicant layer has a second side opposite the first side of the first layer, the first side on the side opposite the wound, wherein the first medicant layer comprises one or more of the following: oxygen-generating composite material and a cerium oxide material.

2. The wound healing dressing of claim 1, wherein wound healing dressing has the characteristics of being anti-inflammatory, an antioxidant, and pro-healing for at least 20 day.

3. The wound healing dressing of claim 1, wherein wound healing dressing has the characteristics of free radical scavenging and oxygen generation.

4. The wound healing dressing of claim 1, wherein cerium oxide material comprises a plurality of cerium oxide nanoparticles.

5. The wound healing dressing of claim 1, wherein the cerium oxide material further comprises at least one biopolymer selected from alginate, hyaluronic acid (HA), chitosan, agarose, collagen, fibrin, gelatin, dextran, and any combination thereof, as well as derivatives of each of these.

6. (canceled)

7. The wound healing dressing of claim 1, wherein the oxygen-generating composite material comprises one or more of the following peroxide materials: calcium peroxide, sodium peroxide, magnesium peroxide, lithium peroxide, potassium peroxide, and a combination thereof.

8. The wound healing dressing of claim 1, wherein the oxygen-generating composite material is a flexible sheet that has a thickness of about 200 to 1000 μm.

9. The wound healing dressing of claim 1, wherein the first medicant layer comprises a plurality of cerium oxide material particles dispersed in a layer of oxygen-generating composite material.

10. The wound healing dressing of claim 1, further comprising a second medicant layer, wherein the second medicant layer as a first side and a second side opposite the first side, wherein the first side of the second medicant layer is adjacent the second side of the first medicant layer, wherein the second medicant layer comprises one or more of the following: the oxygen-generating composite material and the cerium oxide material.

11. The wound healing dressing of claim 1, wherein the first medicant layer includes the oxygen-generating composite material, wherein the second medicant layer comprises the cerium oxide nanoparticles dispersed in the biopolymer.

12. (canceled)

13. A wound healing dressing, comprising a substrate having a first side and a second side on the side opposite the first side, wherein the second side is adjacent the wound, wherein at least a first medicant layer is disposed on the second side of the substrate, wherein the first medicant layer has a first side adjacent the second side of the substrate and the first medicant layer has a second side opposite the first side of the first layer, the second side adjacent the wound, wherein the first medicant layer comprises one or more of the following:
oxygen-generating composite material and a cerium oxide material, wherein the oxygen-generating composite material is a flexible sheet that has a thickness of about 200 to 1000 μm, wherein the flexible sheet has a length of about 1 centimeter to 10 centimeters and a width of about 1 centimeter to 10 centimeters, and wherein the flexible sheet generates greater than about 0.32 mM/day for at least 5 days.

14. The wound healing dressing of claim 13, wherein cerium oxide material comprises a plurality of cerium oxide nanoparticles, wherein the cerium oxide nanoparticle has a longest dimension of about 1 to 20 nanometers.

15. The wound healing dressing of claim 14, wherein the cerium oxide material further comprises at least one biopolymer selected from alginate, hyaluronic acid (HA), chitosan, agarose, collagen, fibrin, gelatin, dextran, and any combination thereof, as well as derivatives of each of these.

16. The wound healing dressing of claim 13, wherein the oxygen-generating composite material comprises one or more of the following peroxide materials: calcium peroxide, sodium peroxide, magnesium peroxide, lithium peroxide, potassium peroxide, and a combination thereof.

17. The wound healing dressing of claim 16, wherein the oxygen-generating composite material further comprises a support material, wherein the support materials comprises: organosilicones, poly(ethersulfone), poly(ethylene oxide terephthalate) block copolymers, polysulfone, and combinations thereof.

18. A wound healing dressing, comprising a substrate having a first side and a second side on the side opposite the first side, wherein the second side is adjacent the wound, wherein a first medicant layer is disposed on the second side of the substrate, wherein the first medicant layer has a first side adjacent the second side of the substrate and the first medicant layer has a second side opposite the first side of the first layer, wherein the substrate includes a second medicant layer, wherein the second medicant layer has a first side and a second side opposite the first side, wherein the first side of the second medicant layer is adjacent the second side of the first medicant layer, wherein the first medicant layer comprises one or more of the following: an oxygen-generating composite material and a cerium oxide material, and wherein the second medicant layer comprises one or more of the following: the oxygen-generating composite material and the cerium oxide material.

19. The wound healing dressing of claim 18, wherein the first medicant layer comprises a plurality of cerium oxide material dispersed in a layer of oxygen-generating composite material, and wherein the second medicant layer comprises the cerium oxide nanoparticles dispersed in a biopolymer.

20. (canceled)

21. The wound healing dressing of claim 18, wherein the first medicant layer includes the oxygen-generating composite material, wherein the oxygen-generating composite material includes a peroxide material, a support material or both; and the second medicant layer includes the cerium oxide material and a biopolymer.

22. The wound healing dressing of claim 19, wherein the first medicant layer is a flexible sheet that has a thickness of about 200 to 1000 μm, wherein the flexible sheet has a length of about 1 centimeter to 10 centimeters and a width of about 1 centimeter to 10 centimeters.

23. The wound healing dressing of claim 19, wherein the first medicant layer and the second medicant layer form a flexible sheet that has a thickness of about 200 to 1000 μm, wherein the flexible sheet has a length of about 1 centimeter to 10 centimeters and a width of about 1 centimeter to 10 centimeters.

24-26. (Canceled)
